(19) United States
(12) Patent Application Publication
(10) Pub. No.: US 2007/0092963 A1
(43) Pub. Date: Apr. 26, 2007

(54) DEVICE TO THE PREPARATION OF CUTTING EDGE CAPABLE SAMPLES

(75) Inventors: Steffen Kraus, Wiesloch (DE); Erwin Schneider, Nussloch (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: PFM PRODUKTE FUR DIE MEDIZIN AG

(21) Appl. No.: 11/470,071

(22) Filed: Sep. 5, 2006

(30) Foreign Application Priority Data
Sep. 5, 2005 (DE) 102005042262.4

(51) Int. Cl. C12M 1/34

(52) U.S. Cl. 435/287.1

(57) ABSTRACT

A device for the processing of samples which can be cut, in particular histological tissue samples, where the device comprises a processing tray (1) with a processing surface (2) and with at least two trimming inserts (3) for laying in and cutting the samples by means of knives, is characterized by the fact that the trimming inserts (3) have an encircling edge (4), which serves for the application of the knife and for the flush cutting of the sample and that the trimming inserts (3) are embodied to have different depths for producing samples of different thicknesses.

The invention relates to a device for the processing of samples which can be cut, in particular histological tissue samples, where the device comprises a processing tray with a processing surface and with at least two trimming inserts for laying in and cutting the samples by means of knives.

A generic device is known from U.S. 2002/0020276 A1. Trimming inserts provided there comprise an elaborate mechanism, according to which the depth of the trimming inserts can be varied from beneath the processing tray by means of a positioning screw. A knife serving for cutting is guided through a slot in a guide with edges at the sides and under a guide rod so that the thickness of the sample is dependent on the setting in the trimming insert on the one hand and on the slot-like guide next to the guide rod on the other hand.

The generic device is elaborate in its construction and, as follows in turn from its elaborate construction, complicated in application. Furthermore, the device comprises countless components with edges, joints, undercuts, etc., to which contamination can adhere. The cleaning effort resulting from this is significant.

The objective of the present invention is to develop and extend the generic device for the processing of samples which can be cut, in particular histological tissue samples, in such a manner that simple handling, including cleaning of the device, is possible with the simplest construction.

The above objective is realized by a device with the features of claim 1. According thereto, the generic device is characterized by the fact that the trimming inserts have an encircling edge which serves for the application the knife and for the flush cutting of the sample and that the trimming inserts are embodied to have different depths for producing samples of different thicknesses.

It has been recognized that the complicated embodiment according to the state of the art is associated with quite significant problems in practice, and in fact, for one thing, in its handling during processing and, for another thing, in cleaning. Both disadvantages result from the elaborate, and thus complicated, construction of the generic device.

Furthermore, it has been recognized that the disadvantages of the generic device can be eliminated with simple means, namely by providing trimming inserts with relief of different depths so that, corresponding to the different depths of the trimming inserts, samples of different thicknesses can be produced. Serving to define the thickness of the sample to be produced, are, on the one hand, the depth of the respective trimming insert and, on the other hand, the encircling, plane edge of the trimming insert which serves for the application of the knife and thus for flush cutting of the sample.

So that samples of different thicknesses can be produced, it is of advantage if the device comprises three to ten, preferably different, trimming inserts, e.g., five trimming inserts. The trimming inserts can have depths of, for example, 1.0 mm, 1.5 mm, 2.0 mm, 2.5 mm, and/or 3.0 mm. Furthermore, it is conceivable that, for the trimming inserts, preferably form-locking inlays to define their spacing are provided. These inlays could be embodied in the form of small disks or plates with a thickness of 0.2 mm or 0.3 mm. Therewith, the greatest possible degree of flexibility is provided. For laying the inlays, a pocket or recess in the processing surface can be provided.

Working with the device according to the invention can be simplified by the trimming inserts being embedded in the processing surface in such a manner that they are adjacent to one another, preferably spaced from one another. Preferably, the trimming inserts are disposed along a peripheral area of the processing tray. With this, adjacent samples of different thicknesses can be produced, and in fact with a cut away from the different trimming inserts in each case.

In principle, it is conceivable that the encircling edge of the trimming inserts which serves for the application, and thus the guiding, of the knife could be shaped so as to be flush with the processing surface. However, for better handling of the knife it is of advantage if the encircling edge of the trimming inserts is raised, at least slightly, with respect to the processing surface so that during cutting the knife has no contact with the processing surface around the trimming inserts.

It is of advantage, in particular for the cleaning of the trimming inserts, if they can be removed from the processing tray. For this purpose it is in turn of advantage if passages for pressing out the trimming inserts are formed in the processing tray beneath the respective trimming inserts so that the trimming inserts can be pressed out upwards from beneath the processing tray.

In principle, it is conceivable that the trimming inserts can have any form. A round structure is likewise conceivable, as is a rectangular embodiment. Furthermore, it is possible that the trimming inserts, with their different depths, have surfaces of the same size or also surfaces of the different sizes. At least one of the trimming inserts could be shaped and dimensioned in such a manner that a commercially available embedded cassette to receive the sample can be laid in. Such a precaution has the great advantage that the sample can already have been cut to its corresponding thickness in the cassette. After removal of the cassette, additional treatments can preferably be carried out automatically.

For easier removal of the embedded cassette the trimming insert has depressions in its peripheral area into which one can reach with one's fingers or with a tool and thus easily remove the embedded cassette. Discretionary developments relating to this are conceivable. Furthermore, it is of advantage if the trimming inserts are made of stainless steel. Such a development is beneficial for handling and cleaning.

In reference to the preparation of the tools serving for handling, it is of advantage if at least one stamp-like holding device for positioned holding of the sample in the trimming insert is assigned to the processing tray. The holding device preferably comprises a rod serving for holding and, serving to press the sample down, a surface which can be embodied to be round or rectangular. From this, the stamp-like embodiment follows.

In reference to the precaution of holding devices it is of additional advantage if two or more holding devices with active surfaces of different sizes, and in given cases different forms, are provided. The holding devices can be made of stainless steel or of plastic.

To prepare the holding devices, receptacles are formed in an advantageous manner in the processing surface and thus in the processing tray. The receptacles for the holding devices can be provided adjacent to one another, preferably at the processing tray's peripheral area opposite the trimming inserts, so that a comprehensible arrangement for comfortable handling is realized.

In the vicinity of the trimming inserts, preferably parallel to the trimming inserts, a scale can be formed on or in the processing surface. The scale is advantageously made of metal, preferably of stainless steel, and can be removed for the purposes of cleaning and replaced for changing the scale units.

Preferably in the central area of the processing tray a trimming adapter can be provided as a working surface for smaller cutting work. This is a type of base which is of preferably high-quality plastic and which is replaceable. Smaller cutting work can thus be carried out in the central area without the surrounding surface of the processing tray being affected or worn. The trimming adapter is preferably embedded flush into the processing surface and can, like the trimming inserts, be ejected upwards from beneath the processing tray.

Furthermore, it is of advantage if in the processing tray a tool area with receptacles for different tools is provided. There, different tools for the processing of the samples are disposed, where the possibility presents itself that the tool area is graded with respect to the processing surface and thus is at least slightly raised from the processing surface. If it is assumed that most people are right-handed, then it is of additional advantage if the tool area is formed to the right of the trimming inserts and of the trimming adapter. It is entirely conceivable to realize devices for right-handed people and devices for left-handed people, where in the case of a development for left-handed people the tool area should be formed to the left of the trimming inserts and of the trimming adapter.

Furthermore, it is of advantage if the tool area comprises a drain opening passing through the processing tray. Thus, it is, for example, conceivable to rinse off tools which have been put down there, where the fluid serving for cleaning or disinfection can escape through the drain opening. Connection to a drain or into a container, possibly via a filter, is conceivable.

The receptacles serving as a place to put down the tools are matched to the different tools and their geometry. Knives, forks, shears, tweezers, etc. serve as tools. Any tools can be added to the basic kit, where the receptacles for the tools can also be shaped to be completely universal so that a certain bandwidth of similar tools can be inserted into the receptacles.

In an additional advantageous manner, preferably in the outer area, a drainage channel surrounding, preferably encircling, the processing surface is provided. Also, the drainage channel can comprise drain openings which are formed as passages through the processing tray. Thus, any fluids are trapped in the drainage channel and arrive through the drain openings in a well-directed manner in an area beneath the processing tray.

The processing tray itself can be made of different materials, where the use of a high-quality plastic is particularly suitable. The plastic is advantageously of a white color so that for the eye of the operator a sufficiently good contrast with respect to the usual tissue samples results.

In connection with the explanation of a preferred embodiment example of the processing device with the aid of the drawing, preferred developments and extensions will also be explained. Shown in the drawing is

the single FIGURE: the device in a schematic, perspective view.

The single figure shows the device for the processing of samples which can be cut, which are not shown in the figure. The samples are, in particular, histological tissue samples. The device comprises a processing tray 1 with a processing surface 2 and with five trimming inserts 3. The trimming inserts 3 serve for laying in and cutting the samples by means of knives.

The five trimming inserts 3 have, seen from left to right, an increasing depth, namely for producing samples of different thicknesses. Furthermore, each of the trimming inserts 3 has an encircling edge 4, which serves for the application of the knife and for the flush cutting of the sample.

Furthermore, the single figure allows it to be seen that an embedding cassette 5 is laid in the outer trimming insert on the right side so that the sample can be positioned in the embedding cassette 5 and cut according to the depth of the trimming insert 3 and the embedding cassette 5. After the cutting, the sample can be removed together with the embedding cassette 5 and passed on for further processing. The trimming insert 3 serving for laying in the embedding cassette 5 comprises depressions 6 so that the embedding cassette 5 can be removed more easily.

The trimming inserts 3 are embodied in rectangular form. The encircling edge 4 of the trimming inserts 3 is raised, at least slightly, with respect to the processing surface 2 so that contact of the knife with the processing surface 2 is essentially ruled out.

In the embodiment example of a device according to the invention and shown in the single figure, two stamp-like holding devices 7 are provided, whose active surfaces are shaped in the form of a circle. The holding devices 7 are positioned in corresponding recesses in the processing tray 1 and serve to hold the sample to be cut in the trimming insert 3.

Furthermore, a scale 8 is provided above the trimming insert 3 so that a direct measurement of the sample is possible.

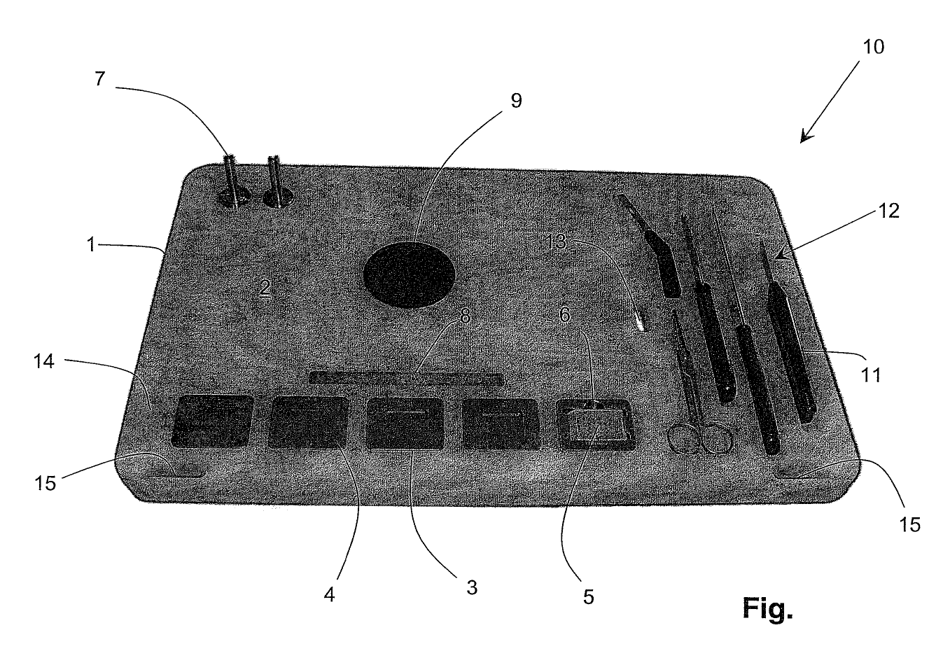

The single FIGURE 1 shows furthermore that processing tray 1 comprises in its central area a trimming adapter 9 as a working surface for smaller cutting work, where the trimming adapter 9 consists of black, high-quality plastic and is set flush in the processing surface 2 or embedded there. The trimming adapter 9 is replaceable and has a circular working surface.

On the right side of the processing tray 1 a tool area 10 is provided into which different tools 11 are inserted. Special receptacles 12 for inserting the tools 11 serve for this purpose. Furthermore, the tool area 10 comprises a drain opening passing through the processing tray 1 for draining off any fluids and materials.

Furthermore, the single figure shows that in the processing tray 1, in the outer area there, a drainage channel 14 entirely encircling the processing surface 2 is provided. In the corner areas a total of four discharge openings 15 are provided in the drainage channel 14, which are formed as passages through the processing tray 1.

In reference to features which cannot be inferred from the single figure, let reference be made to the general part of the description to avoid repetition.

1. A device for the processing of samples which can be cut, in particular histological tissue samples, where the device comprises a processing tray (1) with a processing surface (2) and with at least two trimming inserts (3) for laying in and cutting the samples by means of knives, characterized by the fact that the trimming inserts (3) have an encircling edge (4), which serves for the application of the knife and for the flush cutting of the sample and that the trimming inserts (3) are embodied to have different depths for producing samples of different thicknesses.

2-38. (canceled)
